(19) United States
(12) Patent Application Publication
(10) Pub. No.: US 2026/0232295 A1
(43) Pub. Date: Aug. 13, 2026

(54) SYSTEM AND METHODS FOR AUTONOMOUS ULTRASONOGRAPHIC IMAGING

(71) Applicants: Ohio State Innovation Foundation, Columbus, OH (US); Arizona Board of Regents on Behalf of the University of Arizona, Tucson, AZ (US)

(72) Inventors: Alper YILMAZ, Columbus, OH (US); Srikar ADHIKARI, Tucson, AZ (US)

(21) Appl. No.: 19/469,578

(22) PCT Filed: Mar. 26, 2024
(86) PCT No.: PCT/US2024/021460

§ 371 Date: Sep. 26, 2025

Related U.S. Application Data
(60) Provisional application No. 63/492,326, filed on Mar. 27, 2023.

(51) Int. Cl. A61B 8/00; A61B 8/10; B06B 1/06; G06T 7/00

(52) U.S. Cl. CPC A61B 8/463; A61B 8/10; A61B 8/4209; A61B 8/4488; A61B 8/54; B06B 1/067; B06B 1/0681; G06T 7/0012; B06B 2201/76; G06T 2200/24; G06T 2207/10132; G06T 2207/20084; G06T 2207/30041

(57) ABSTRACT

An autonomous ultrasonographic imaging device includes an ultrasound transducer array, a processor, and memory having instructions stored thereon that, when executed by the processor, cause the autonomous ultrasonographic imaging device to capture a plurality of ultrasound images of a target area of a subject using the ultrasound transducer array, evaluate the plurality of ultrasound images using an artificial intelligence model to detect a medical condition, and display an indication of whether the medical condition was detected via a user interface. In some implementations, the autonomous ultrasonographic imaging device includes an electrotechnical movement assembly that can incrementally adjust a positioning of the ultrasound transducer array, such as between captures of the plurality of ultrasound images.

### CROSS-REFERENCE TO RELATED APPLICATION

This application claims priority to U.S. Provisional Patent Application No. 63/492,326, filed Mar. 27, 2023, entitled, “SYSTEM AND METHODS FOR AUTONOMOUS ULTRASONOGRAPHIC IMAGING,” the disclosure of which is incorporated herein by reference in its entirety.

### STATEMENT REGARDING FEDERALLY SPONSORED RESEARCH OR DEVELOPMENT

This invention was made with government support under W81XWH2210803 awarded by the US Army Medical Research and Development Command. The government has certain rights in the invention.

### BACKGROUND

Ultrasound is a common diagnostic imaging tool used to evaluate various medical conditions. Acquisition of ultrasound images is generally performed by trained sonographers and requires hand-eye coordination for performing rotation and translation maneuvers to capture quality images. Techniques for capturing quality ultrasound images are traditionally mastered over time through practice by sonographers. However, there remains an unmet need for ultrasound technologies to aid inexperienced healthcare providers in acquiring and interpreting ultrasonographic images.

### SUMMARY

One implementation of the present disclosure is an autonomous ultrasonographic imaging device including: an ultrasound transducer array; a processor; and memory having instructions stored thereon that, when executed by the processor, cause the autonomous ultrasonographic imaging device to: capture, using the ultrasound transducer array, a plurality of ultrasound images of a target area of a subject; evaluate the plurality of ultrasound images using an artificial intelligence (AI) model to detect a medical condition; and display, via a user interface, an indication of whether the medical condition was detected.

some implementations, the autonomous ultrasonographic imaging device further includes an electromechanical movement assembly, wherein the ultrasound transducer array is coupled to the electromechanical movement assembly such that the positioning of the ultrasound transducer array can be adjusted.

In some implementations, the autonomous ultrasonographic imaging device further includes a housing, wherein the ultrasound transducer array, the electromechanical movement assembly, the processor, and the memory are at least partially enclosed within the housing.

In some implementations, to capture the plurality of images, the instructions further cause the autonomous ultrasonographic imaging device to control the electromechanical movement assembly to incrementally adjust the positioning of the ultrasound transducer array between captures of each of the plurality of ultrasound images.

In some implementations, the electromechanical movement assembly is configured to incrementally rotate the ultrasound transducer array about an axis between captures of each of the plurality of ultrasound images to generate a rotating cross-section of the target area of the subject.

In some implementations, the electromechanical movement assembly is configured to incrementally rotate the ultrasound transducer array at an interval of between one (1) and five (5) degrees.

In some implementations, the user interface includes a control indicator light and a display.

In some implementations, to display the indication of whether the medical condition was detected: the control indicator light is turned on and the display is turned on if the medical condition is detected; the control indicator light is turned on and the display is turned off if the medical condition is not detected; or the control indicator light is turned off and the display is turned on if results of the evaluation of the plurality of images are determined to be invalid.

In some implementations, the user interface further includes a speaker for providing audible feedback to an operator.

In some implementations, the instructions further cause the autonomous ultrasonographic imaging device to output a sound via the speaker when the medical condition is detected or once the evaluation of the plurality of ultrasound images is complete.

In some implementations, the AI model is a machine learning model.

In some implementations, the machine learning model is a deep learning model.

In some implementations, the deep learning model is a convolutional neural network.

In some implementations, the deep learning model comprises a feature extraction module, a convolutional layer module, and a transformer module.

In some implementations, the machine learning model is a reinforcement learning model.

In some implementations, the reinforcement learning is a deep reinforcement learning model with neural network as its policy.

In some implementations, the autonomous ultrasonographic imaging device is configured for ocular ultrasound imaging and is worn or positioned over an eye of the subject.

In some implementations, plurality of images are images of an optical nerve sheath.

In some implementations, the medical condition is a mild traumatic brain injury (mTBI), and wherein the plurality of ultrasound images are evaluated, in part, to determine an optical nerve sheath diameter (ONSD).

In some implementations, the autonomous ultrasonographic imaging device is configured to be worn on a chest of the subject.

In some implementations, the ultrasound transducer array includes at least one ultrasound transducer, the at least one ultrasound transducer including a lens, a matching layer, a ground electrode, a piezoelectric ceramic layer, a signal electrode, and an acoustic absorption layer.

In some implementations, the instruction further causing the autonomous ultrasonographic imaging device to receive programming instructions from a remote device using via an application programming interface (API), wherein the programming instructions define the medical condition that the plurality of images are evaluated to detect and/or include the AI model.

Another implementation of the present disclosure is a method of detecting medical conditions using an autonomous ultrasonographic imaging device, the method including: capturing, by an ultrasound transducer array of the autonomous ultrasonographic imaging device, a plurality of ultrasound images of a target area of a subject, wherein a position of the ultrasound transducer array is automatically and incrementally adjusted between captures of each of the plurality of images; evaluating the plurality of ultrasound images using am artificial intelligence (AI) model to detect a medical condition; and displaying, via a user interface of the autonomous ultrasonographic imaging device, an indication of whether the medical condition was detected.

In some implementations, the position of the ultrasound transducer array is automatically and incrementally adjusted by an electromechanical movement assembly, wherein the ultrasound transducer array is coupled to the electromechanical movement assembly.

In some implementations, capturing the plurality of images further includes controlling the electromechanical movement assembly to automatically and incrementally adjust the positioning of the ultrasound transducer array.

In some implementations, the electromechanical movement assembly is configured to incrementally rotate the ultrasound transducer array about an axis between captures of each of the plurality of ultrasound images to generate a rotating cross-section of the target area of the subject.

In some implementations, the electromechanical movement assembly is configured to incrementally rotate the ultrasound transducer array at an interval of between one (1) and five (5) degrees.

In some implementations, the user interface includes a control indicator light and a display.

In some implementations, displaying the indication of whether the medical condition was detected includes: turning on the control indicator light and the display if the medical condition is detected; turning on the control indicator light and turning off the display if the medical condition is not detected; or turning off the control indicator light and turning on the display if results of the evaluation of the plurality of images are determined to be invalid.

In some implementations, the user interface further includes a speaker for providing audible feedback to an operator.

In some implementations, the method further includes outputting a sound via the speaker when the medical condition is detected or once the evaluation of the plurality of ultrasound images is complete.

In some implementations, the autonomous ultrasonographic imaging device is configured for ocular ultrasound imaging and is worn or positioned over an eye of the subject.

In some implementations, plurality of images are images of an optical nerve sheath.

In some implementations, the medical condition is a mild traumatic brain injury (mTBI), and wherein the plurality of ultrasound images are evaluated, in part, to determine an optical nerve sheath diameter (ONSD).

In some implementations, the autonomous ultrasonographic imaging device is configured to be worn on a chest of the subject.

In some implementations, the ultrasound transducer array includes at least one ultrasound transducer, the at least one ultrasound transducer including a lens, a matching layer, a ground electrode, a piezoelectric ceramic layer, a signal electrode, and an acoustic absorption layer.

In some implementations, the method further includes receiving programming instructions from a remote device using via an application programming interface (API), wherein the programming instructions define the medical condition that the plurality of images are evaluated to detect and/or include the AI model.

Additional features will be set forth in part in the description which follows or may be learned by practice. The various features described herein may be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1 is a block diagram of an autonomous ultrasonographic imaging device, according to some implementations.

FIG. 2 is a flow chart of a process for detecting medical conditions using the autonomous ultrasonographic imaging device of FIG. 1, according to some implementations.

FIG. 3 is example architecture for biomedical deep learning applications, according to some implementations.

FIG. 4 illustrates the Grouped Atrous Spatial Pyramid Pooling Module of FIG. 3 in greater detail, according to some implementations.

FIG. 5 is a diagram of an example ultrasound transducer array, according to some implementations.

FIG. 6 is a diagram illustrating movement of the ultrasound transducer array of FIG. 5 during imaging, according to some implementations.

FIG. 7 is a diagram of various example user interfaces, according to some implementations.

Various objects, aspects, and features of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a fast, non-invasive, and objective diagnostic machine-learning-based autonomous ultrasonographic imaging system and corresponding methods are shown, according to various implementations. In at least one aspect, a portable and/or wearable autonomous ultrasonographic imaging device is disclosed, which uses lightweight machine-learning models to quickly evaluate captured ultrasound images for the detection of various medical conditions. The ultrasonographic imaging device disclosed herein generally includes an ultrasound transducer array positioned on an electrotechnical movement assembly, such that a position of the ultrasound transducer array can be automatically and/or incrementally adjusted as the device captured ultrasound images. In this way, a cross-section of a target area of a subject (e.g., an injured person) can be generated and evaluated, resulting in a more robust image set and more accurate diagnostic results.

Notably, the disclosed system and methods can be used on-sight, in the field, or at a point of injury to quickly evaluate various medical conditions. In this regard, the need for urgent transfer and treatment can be determined to significantly impact mortality, long-term outcomes, and healthcare costs. The medical cases for diagnosis and monitoring includes but is not limited to traumatic hemopericardium, retinal detachment, mild traumatic brain injury (mTBI), conditions of the liver, and the like. As mentioned above, the skill level of a healthcare provider, first responder, or other caretaker for an injured subject is the principal limitation in the use of point-of-care ultrasonography. The disclosed system and method address this limitation by leveraging machine learning capabilities to acquire and interpret ultrasonographic images. Machine learning algorithms can mitigate challenges with training and expertise by providing inexperienced users with reliable automated tools capable of detecting ultrasonographic signs of medical conditions stated above. This technology has the potential to transform the care in communities with limited access to healthcare and in the battlefield by enabling non-experts to use ultrasonographic devices to detect medical conditions.

With these considerations, it is critical to develop a noninvasive, hands-free, wearable ultrasonographic imaging device that could accurately detect medical conditions and continuously monitor them to guide interventions and improve survival and recovery. By integrating machine-learning-assisted image acquisition and analysis with wearable technology, it will be possible for inexperienced personnel in resource-limited settings to use the disclosed ultrasonographic imaging device and direct appropriate therapy, while also allow healthcare providers to focus on other aspects of resuscitation. A wearable device on the chest, eye, or other body areas with continuous ultrasonographic capabilities will also be a valuable hemodynamic tool to monitor patients with various medical conditions in the civilian and defense world.

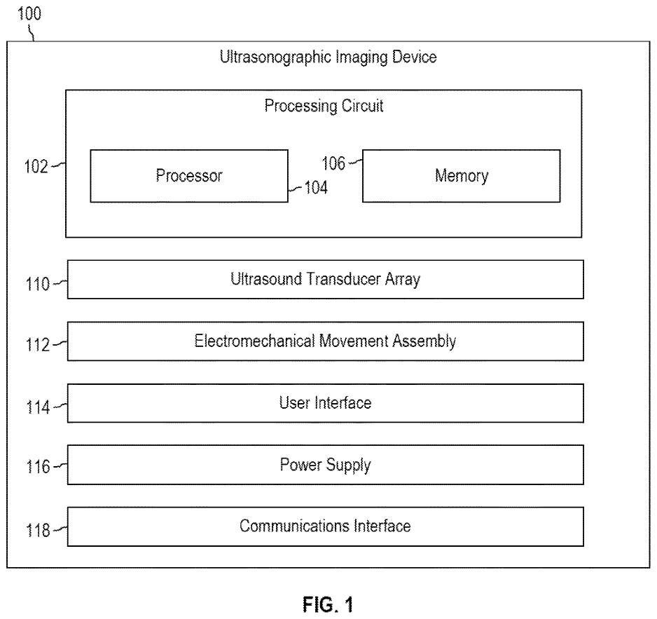

Referring to FIG. 1, a block diagram of an autonomous ultrasonographic imaging device 100 is shown, according to some implementations. Generally, ultrasonographic imaging device 100 is portable and/or wearable; thus, with respect to the following description, it should be understood that the physical dimensions of ultrasonographic imaging device 100 and the components thereof are sized to be worn and/or carried by a person. In some implementations, ultrasonographic imaging device 100 is configured to be worn across the chest of a subject. In some implementations, ultrasonographic imaging device 100 is configured to be worn on the head and positioned across the subject's eye for ocular imaging. In general, ultrasonographic imaging device 100 may be generally configured to implement or execute the various processes and methods described herein.

Ultrasonographic imaging device 100 generally includes a processing circuit 102 that includes a processor 104 and a memory 106. Processor 104 can be a general-purpose processor, an application specific integrated circuit (ASIC), one or more field programmable gate arrays (FPGAs), a group of processing components, or other suitable electronic processing structures. In some implementations, processor 104 is configured to execute program code stored on memory 106 to cause ultrasonographic imaging device 100 to perform one or more operations, as described below in greater detail. It will be appreciated that, in implementations where ultrasonographic imaging device 100 is part of another computing device (e.g., a general-purpose computer), the components of ultrasonographic imaging device 100 may be shared with, or the same as, the host device.

Memory 106 can include one or more devices (e.g., memory units, memory devices, storage devices, etc.) for storing data and/or computer code for completing and/or facilitating the various processes described in the present disclosure. In some implementations, memory 106 includes tangible (e.g., non-transitory), computer-readable media that stores code or instructions executable by processor 104. Tangible, computer-readable media refers to any physical media that is capable of providing data that causes ultrasonographic imaging device 100 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 106 can include RAM, ROM, hard drive storage, temporary storage, non-volatile memory, flash memory, optical memory, or any other suitable memory for storing software objects and/or computer instructions. Memory 106 can include database components, object code components, script components, or any other type of information structure for supporting the various activities and information structures described in the present disclosure. Memory 106 can be communicably connected to processor 104, such as via processing circuit 102, and can include computer code for executing (e.g., by processor 104) one or more processes described herein.

While shown as individual components, it will be appreciated that processor 104 and/or memory 106 can be implemented using a variety of different types and quantities of processors and memory. For example, processor 104 may represent a single processing device or multiple processing devices. Similarly, memory 106 may represent a single memory device or multiple memory devices. Additionally, in some implementations, ultrasonographic imaging device 100 may be implemented within a single computing device (e.g., one housing, etc.). In other implementations, ultrasonographic imaging device 100 may be distributed across multiple computers (e.g., that can exist in distributed locations). For example, ultrasonographic imaging device 100 may include multiple distributed computing devices (e.g., multiple processors and/or memory devices) in communication with each other that collaborate to perform operations. In some such implementations, certain functions of ultrasonographic imaging device 100, as described herein, can be executed or performed by a remote computing device. For example, ultrasonographic imaging device 100 may be configured to capture ultrasound images but may transmit the images to a remote computing device (e.g., another computer, a server, etc.) for processing.

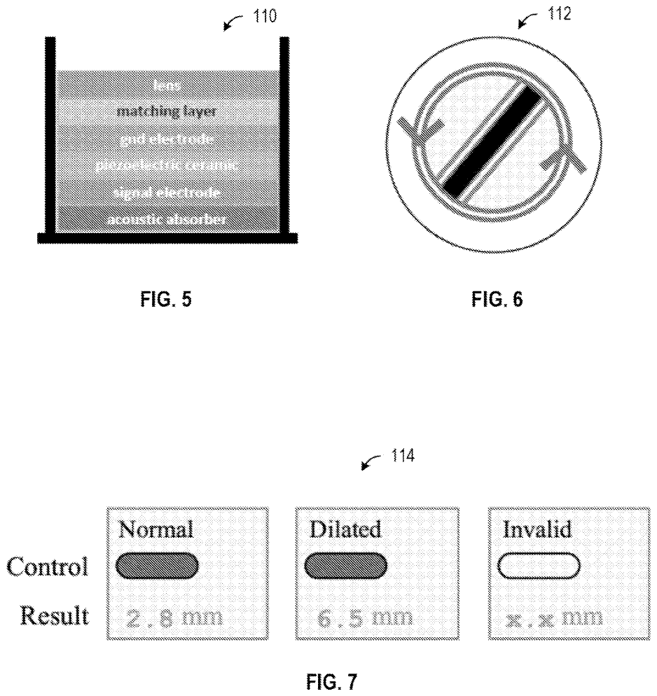

Ultrasonographic imaging device 100 is further shown to include an ultrasound transducer array 110, which generally includes one or more ultrasound transducers. In some implementations, ultrasound transducer array 110 includes a single ultrasound transducer. In some implementations, each transducer of ultrasound transducer array 110 is formed of a lens, a matching layer, a ground (gnd) electrode, a piezoelectric ceramic layer, a signal electrode, and an acoustic absorption material layer, as shown in FIG. 5; however, it should be understood that the specific configuration and construction of ultrasound transducer array 110 is not intended to be limiting. Ultrasound transducer array 110 is generally configured to capture ultrasound images of a target area of a subject. In some implementations, ultrasound transducer array 110 is controlled by processing circuit 102 and, accordingly, may transmit captured images to processing circuit 102 for processing.

In some implementations, ultrasound transducer array 110 is positioned on and/or coupled to an electromechanical movement assembly 112. As described herein, electromechanical movement assembly 112 generally includes one or more electromechanical components (e.g., motors, levers, gears, actuators, arms, brackets, etc.) that can be electronically controlled by processing circuit 102 to produce movement. Specifically, electromechanical movement assembly 112 may be controlled by processing circuit 102 in order to manipulate/adjust a positioning of ultrasound transducer array 110. In some implementations, electromechanical movement assembly 112 is configured to change the position of ultrasound transducer array 110 incrementally as ultrasound images are captured. In some implementations, electromechanical movement assembly 112 is configured to have a single degree of freedom. As shown in FIG. 6, for example, electromechanical movement assembly 112 may be configured to incrementally rotate ultrasound transducer array 110 about an axis of rotation. If configured for ocular imaging (e.g., of a subject's eye), ultrasound transducer array 110 and electromechanical movement assembly 112 may thus cooperatively generate rotating cross-sections of the eye anatomy that can be used to measure optical nerve sheath diameter (ONSD).

Cooperatively, ultrasound transducer array 110 and electromechanical movement assembly 112 can capture a plurality of ultrasound images of a target area of a subject at various positions. To this point, ultrasound transducer array 110 and electromechanical movement assembly 112 may be controlled by processing circuit 102 to initiate the capture of said images. Subsequently, the captured images may be sent to processing circuit 102 for processing using a machine-learning model. The model may evaluate the series of images to detect a medical condition, such as mTBI. In some implementations, electromechanical movement assembly 112 is configured to incrementally rotate ultrasound transducer array 110, such as in increments of one to five degrees, to generate a cross-section of the target area of the subject. In this manner, untrained operators are able to capture quality ultrasound images without mastering rotation and translation maneuvers, as typically required with traditional ultrasound machines.

In some implementations, ultrasonographic imaging device 100 includes a user interface 114 for presenting/displaying results of the evaluation of captured ultrasound images. In some implementations, user interface 114 includes a control indicator and/or a display. In some implementations, the controller indicator is a light (e.g., LED) and results or additional data are presented via the display. In other implementations, the control indicator and display are integrated. Considering heating and battery bandwidth issues, in some implementation, user interface 114 may take the form shown in FIG. 7. User interface 114 may, in particular, show the quality of acquired images as ‘control’ and results of evaluating the captured ultrasound images. In the context of ocular sonography, the ‘results’ field may show ONSD measurements. In some implementations, user interface 114 includes a speaker for outputting sound.

In some implementations, ultrasonographic imaging device 100 includes a power supply 116 configured to power any of the components described herein. Power supply 116 may be a battery, a capacitor, a collection of energy storage components, or may include a connector for electrical connection to an external power source. In some implementations, power supply 116 is a lithium-ion battery or other type of rechargeable battery suitable to power the various components of ultrasound transducer array 110 as described herein.

In some implementations, ultrasonographic imaging device 100 includes a communications interface 118 that facilitates communications between ultrasonographic imaging device 100 and any external components or devices (e.g., computers). Accordingly, communications interface 118 can be, or can include, any number of wired or wireless communications interfaces (e.g., jacks, antennas, transmitters, receivers, transceivers, wire terminals, etc.) for conducting data communications, or a combination of wired and wireless communication interfaces. In various implementations, communications via communications interface 118 are direct (e.g., local wired or wireless communications) or via a network (e.g., a WAN, the Internet, a cellular network, etc.). For example, communications interface 118 may include one or more Ethernet ports for communicably coupling ultrasonographic imaging device 100 to a network (e.g., the Internet). In another example, communications interface 118 can include a Wi-Fi transceiver for communicating via a wireless communications network. In yet another example, communications interface 118 may include cellular or mobile phone communications transceivers or short-range wireless transceivers (e.g., Bluetooth®).

While not shown in FIG. 1, it should be appreciated that ultrasonographic imaging device 100 can also include a housing which contains all or at least some of the components described herein. For example, the housing may at least partially enclose processing circuit 102, ultrasound transducer array 110, electromechanical movement assembly 112, power supply 116, and communications interface 118. In some implementations, user interface 114 is positioned on the housing so as to be visible to an operator of ultrasonographic imaging device 100. In some implementations, the housing is formed of plastic or another suitable material. The housing may also be formed in an ergonomic shape for handling by an operator and/or wearing by a subject. In some implementations, the housing is formed of a non-allergenic material.

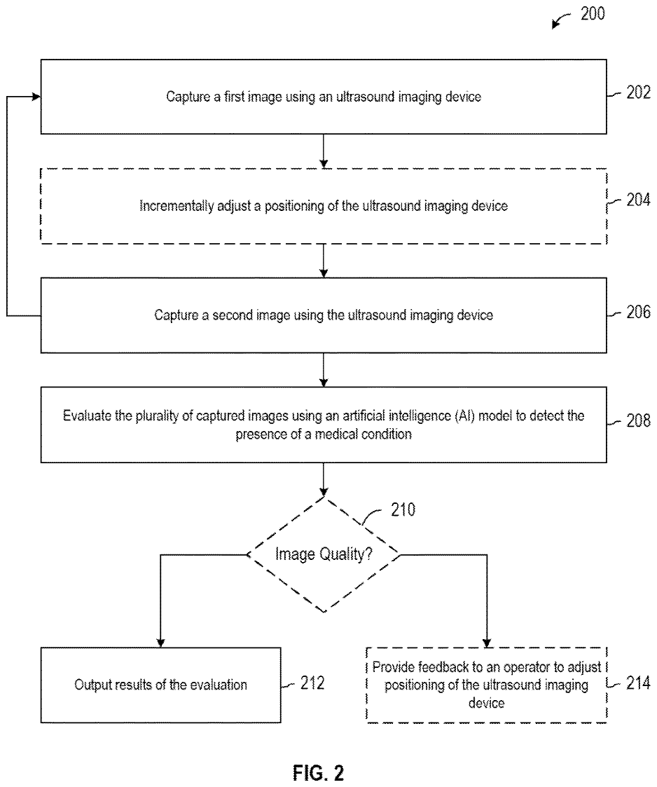

For added clarity, consider an example use-case where ultrasonographic imaging device 100 is configured for ocular ultrasonography. Ocular sonography, in this context, generally relates to the measurement of ONSD to detect mTBIs and other types of injuries. During operation of ultrasonographic imaging device 100, when turned on, ultrasound transducer array 110 and electromechanical movement assembly 112 will cooperatively begin acquiring ocular ultrasound images at angular increments. During this phase, both indicators of user interface 114 (e.g., the control LED and results display) will be turned off, denoting that the device is acquiring images. Once a sequence of images is collected, ultrasonographic imaging device 100 will run the deep-learning and three-dimensional (3D) geometric functions discussed Appendix A and below with respect to FIG. 2. The results of this evaluation of the captured images will be shown by turning on the indicators of user interface 114. If the control indicator is ON and result indicator is OFF, the operator will be informed that dilated ONSD (12ndicateve of complicated mTBI) condition is not detected. If both indicators are ON, the operator will be informed that the medical condition is detected. In some cases, there will also be a sound notification. When only the result indicator is ON, the operator will be notified that the result is not valid (e.g., potentially due to poor quality images). The control indicator may be turned ON and OFF based on the completion of the image acquisition task.

Referring now to FIG. 2, a flow chart of a process 200 for detecting medical conditions from ultrasound images is shown, according to some implementations. In some implementations, process 200 is implemented by ultrasonographic imaging device 100, as described above. However, it should be understood that process 200 can, more generally, be implemented or executed by any suitable device (e.g., a non-wearable ultrasound). In some implementations, certain steps of process 200 are performed by a device that is remote from ultrasonographic imaging device 100. For example, ultrasonographic imaging device 100 may only capture ultrasound images, which are then transmitted to a remote device for further processing. It will be appreciated that certain steps of process 200 may be optional and, in some implementations, process 200 may be implemented using less than all of the steps. It will also be appreciated that the order of steps shown in FIG. 2 is not intended to be limiting.

At step 202, a first image is captured using an ultrasound imaging device. With respect to ultrasonographic imaging device 100, the first image is generally captured by ultrasound transducer array 110. At step 204, the positioning of ultrasound transducer array 110 is incrementally adjusted. In some implementations, the positioning of ultrasound transducer array 110 is adjusted by operating electromechanical movement assembly 112. Specifically, in some implementations, electromechanical movement assembly 112 is configured to rotate ultrasound transducer array 110 between one and five degrees about an axis of rotation. At step 206, a first image is captured by ultrasound imaging device. Steps 202-206 may then, optionally, be repeated n number of times to generate/collect a series of ultrasound images. In some implementations, steps 202-206 are completed until electromechanical movement assembly 112 rotates ultrasound transducer array 110 about a full 360 degrees.

At step 208, the plurality of captured ultrasound images are evaluated using an artificial intelligence (AI) model to detect the presence of a medical condition. It should be understood that the AI model is deployed in inference mode at step 208. In other words, the AI model has already been trained on a dataset to detect the presence of the medical condition. The medical condition may be an mTBI, traumatic hemopericardium, retinal detachment, or any other condition that can be detected via ultrasound. In some implementations, ultrasonographic imaging device 100 is trained or reprogrammed to detect different medical conditions. In some implementations, ultrasonographic imaging device 100 is reprogrammed via an application programming interface (API) based on the type of medical condition to be detected. In some such implementations, reprogramming may install a new AI model on ultrasonographic imaging device 100. In any case, the AI model may receive the captured ultrasound images as an input and may output an indication of whether the medical condition is detected. In this implementation, the captured ultrasound images comprise the features input into the AI model, and the detected medical condition is the target.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

In some implementations described herein, the AI model is a machine learning model. For example, the AI model can be a deep learning model. The deep learning model is an artificial neural network such as a convolutional neural network.

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation.

A convolutional neural network (CNN) is a type of deep neural network that has been applied, for example, to image analysis applications. Unlike a traditional neural networks, each layer in a CNN has a plurality of nodes arranged in three dimensions (width, height, depth) CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks. Additionally, in some implementations, the CNN includes atrous convolutional layers (sometimes referred to as a dilated convolutional layer), which introduce a dilation rate parameter, which defines spacing between values in the kernel, to the convolution. Atrous convolutional layers are known in the art and therefore not described in further detail herein.

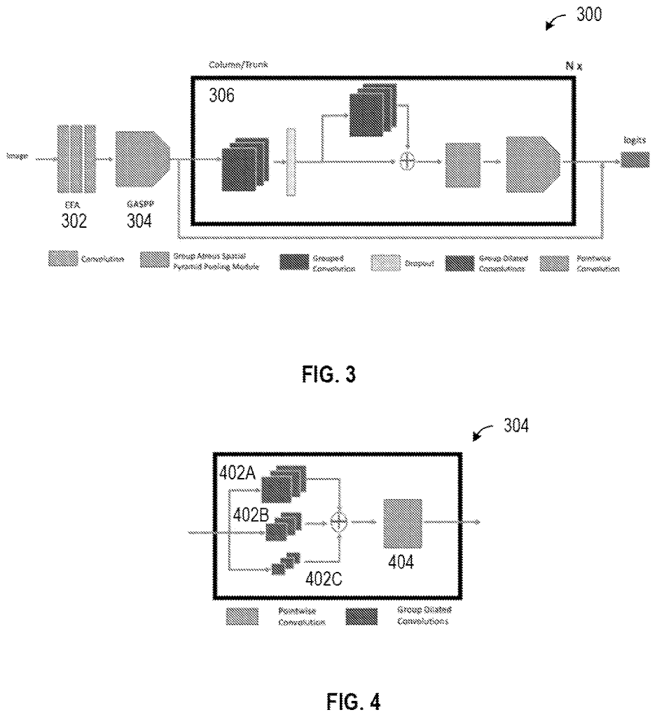

An example deep learning model architecture implemented as a CNN is shown in FIG. 3. The architecture 300, introduced herein as LeavisNet, is an efficient general purpose lightweight architecture developed to perform efficiently in medical imaging tasks. This deep learning model architecture 300 includes an early feature extraction module 302, a convolutional layer module, and a transformer module. The deep learning model architecture 300 is a lightweight model as compared to conventional image classifiers such as ResNet or Inception-v3. For example, the deep learning model architecture 300 is designed to contain about 350,000 parameters, which is less than 10M+ parameters contained in ResNet and Inception-v3.

The Early Feature Extraction (EFA) module 302 is a lightweight preprocessing stage that consists of convolution layers. Depending on the specific input resolution of the EFA module 302 can be modified to control the spatial resolution of the generated features. In some implementations, the EFA module 302 may include three layers: a convolution layer, a GELU nonlinearity followed by a Batch Normalization step. To preprocess the data, the first two convolution layers may include a 3×3 kernel with a stride of 2 and the final convolution layer is a 1×1 convolution with a stride of 2. Additionally, each of the convolution layer may generate 64, 128 and 256 channels during the preprocessing stage. The EFA module 302 increases the dimensionality while learning low and intermediate level features of the input. The reduction in the spatial resolution allows for minimization of the parameter count and the computational load on the downstream modules.

A Grouped Atrous Spatial Pyramid Pooling Module (GASPP) 304 is shown in greater detail in FIG. 4. The GASPP 304 may include three major components: Grouped Convolutions 402A, 402B, 402C; Atrous Convolutions 404; and a Spatial Pyramid Pooling component. At a high level the GASPP module 304 examines an incoming tensor at multiples scales in an efficient manner and produce a compressed representation of the important features. Combining Grouped Atrous Convolution with Spatial Pyramid Pooling allows the model to understand the incoming features with multiple filers of varying fields of views. This allows the model to capture and understand objects as well as contexts at multiple scales without dramatically increasing computational complexity.

Within the GASPP module 304, atrous or dilated convolutions with filter sizes 3, 6 and 9 explicitly model the resolution at which feature responses are computed. The number of channels produced by each convolution layer is fixed to 128 features. Dilated convolutions allow for an efficiently enlarge the field of view of each filter without increasing the number of parameters of the model. By increasing the field of view, the model may obtain a greater understanding of the visual contexts present in each image and how they relate to each other. Grouped convolutions within the GASPP module 304 on the other hand allow for efficient introduction of multiple filters into a single convolution operation which produce multiple channel outputs per layer. This technique allows for learning multiple unique high and low-level features from the same input tensor which otherwise would require multiple successive convolution layers. In addition to efficiency, by exposing a single input tensor to multiple high dimensional transformations the model's representation power can be increased by increasing the possible transformations that can be applied to the input. Finally, grouped atrous/dilated convolutions combines the efficiency of atrous convolutions with the representation power of group convolutions. This combination allows joint learning of multiple unique representations from a single convolution layer by providing additional information from a larger field of view.

Referring again to FIG. 3, a majority of the learnable layers are found within the repeatable column/trunk (RCT) module 306. The RTC module 306 ingests and generates features of the same size allowing the module to be repeatable without affecting spatial resolution of the features. Additionally, it helps simplify the overall complexity of the model since a single parameter can control the depth of the network. The RCT module 306 may consist of five main components which include two grouped convolution stages, a dropout layer, two residual connection points, a pointwise convolution and finally the GASPP module.

The information flow within the RCT module 306 is as follows. An incoming set of features are passed into a grouped convolution layer with a 3×3 filter which computes an initial transformation of the input. Once transformed, a dropout layer reduces the available information present in the learned features prior to an additional group convolution layer. The generated features from the initial and secondary group convolutions are combined to produce an intermediate representation. The modified residual connection mimics and/or introduces a source of noise during training to enforce a robust feature representation. The combined intermediate representation is provided as an input to a pointwise convolution layer which helps learn features across the channels and are passed on to the GASPP is added between the RCT modules.

Thus, the nature of the design of the present disclosure allows the model to easily obtain information from a much larger context by enforcing a fixed spatial resolution within the column/trunk module while simultaneously expanding the field of view within it via dilated convolutions.

Returning to FIG. 2, at step 210, image quality of the captured images may optionally be evaluated, either before or in conjunction with the evaluation at step 208. In some implementations, if the image quality is poor or does not meet a standard (e.g., a threshold), process 200 can continue to step 214, where feedback is provided to an operator (e.g., via user interface 114). Said feedback may provide information or prompts to the operator to adjust positioning of ultrasonographic imaging device 100 and/or to restart the ultrasound capturing process. In other cases, or when the image quality is sufficient to make a prediction of the presence of the medical condition, process 200 can continue to step 212, where an indication of the predict and/or results of the evaluation at step 208 are displayed via a user interface. An example of such a user interface is shown in FIG. 7.

### Experimental Results

To demonstrate the LeavisNet's ability to generate meaningful representations, multiple experiments were performed on binary classification, multiclass classification, and image segmentation. Additionally, data from multiple imaging modalities are used to demonstrate the adaptability of the model to multiple data sources. Table 1, below, shows MedMNISTv2 experiment results. References to (28) and (224) indicate the size of input image. Each column with the experiments indicates the final testing accuracy for each architecture L and S indicates Large and Small Mobilenetv3 models respectively. The data confirms that the LeavisNet architecture 300 demonstrates competitive performance across some of the most prominent models in deep learning literature.

TABLE 1

| | | Path | Organ 5 | Derma | Oct | Blood | Pneu | Retina | Breast | Tissue |
|---|---|---|---|---|---|---|---|---|---|---|
| | | MNIST | MNIST | MNIST | MNIST | MNIST | MNIST | MNIST | MNIST | MNIST |
| Method/Model | Params | Acc | Acc | Acc | Acc | Acc | Acc | Acc | Acc | Acc |
| [symbol] 18(28) | ~11M | [symbol] | 7 [symbol] | 7 [symbol] | 7 [symbol] | [symbol] | [symbol] | 5 [symbol] | 86 [symbol] | 67 [symbol] |
| [symbol] 18(224) | ~11 [symbol] | [symbol] | 77 [symbol] | 7 [symbol] | 76 [symbol] | 9 [symbol] | [symbol] | 49 [symbol] | 8 [symbol] | 6 [symbol] |
| [symbol] (28) | ~ [symbol] | 91.1 | 77 | 7 [symbol] | 76 [symbol] | 9 [symbol] | [symbol] | 5 [symbol] | 81 [symbol] | 68 |
| [symbol] (224) | ~ [symbol] | [symbol] | 7 [symbol] | 7 [symbol] | 77 [symbol] | 95 | 88 [symbol] | 51.1 | 84 [symbol] | 68 |
| Auto [symbol] | — | [symbol] | 67 [symbol] | 7 [symbol] | [symbol] | 87 [symbol] | 8 [symbol] | 51.5 | 80 [symbol] | [symbol] |
| [symbol] | — | 8 [symbol] | 81.3 | 7 [symbol] | [symbol] | 9 [symbol] | [symbol] | 50.3 | 8 [symbol] | 70.3 |
| Google AutoML Vision | — | [symbol] | 7 [symbol] | 7 [symbol] | 77 [symbol] | [symbol] | 94.6 | 53.1 | 86.1 | 67.3 |
| EfficientNet-B0 (28) | ~4M | [symbol] | 7 [symbol] | 7 [symbol] | [symbol] | [symbol] | 87.56 | 58.78 | 79.4 | 70.08 |
| EfficientNet-B0 (224) | ~4M | [symbol] | 7 [symbol] | 78.71 | [symbol] 2.85 | 98.17 | [symbol] | 58 [symbol] | [symbol] | 73.51 |
| MNasNet (28) | ~3.1M | [symbol] | 65 [symbol] | 67 [symbol] | 7 [symbol] | 9 [symbol] | 73.25 | [symbol] | 80.8 | 69.49 |
| MNasNet (224) | ~3.1M | [symbol] | [symbol] | [symbol] | 8 [symbol] | 9 [symbol] | 74 [symbol] | 50.39 | [symbol] | 72.76 |
| MobileNetv3-L (28) | ~4.2M | [symbol] | 57.57 | 7 [symbol] | [symbol] | 9 [symbol] | [symbol] | 57.42 | [symbol] | 67.35 |
| MobileNetv3-L (224) | ~4.2M | 91.69 | 7 [symbol] .68 | 7 [symbol] | 7 [symbol] | 9 [symbol] | [symbol] | [symbol] | 88 [symbol] | 73.47 |
| MobileNetv3-S (28) | ~1.5M | [symbol] | 64. [symbol] | 7 [symbol] | [symbol] | 9 [symbol] | [symbol] | 61.71 | 82 [symbol] | 63.64 |
| MobileNetv3-S (224) | ~1.5M | [symbol] | 71. [symbol] | 7 [symbol] | 8 [symbol] | 9 [symbol] | [symbol] | 58 [symbol] | 8 [symbol] | 73.02 |
| [symbol] (28) | [symbol] | 19 | 45 [symbol] | 7 [symbol] | [symbol] | [symbol] | [symbol] | 50 [symbol] | [symbol] | 32 [symbol] |
| [symbol] (224) | [symbol] | [symbol] | 6 [symbol] | 7 [symbol] | [symbol] 76 | [symbol] | [symbol] | [symbol] | [symbol] | 54 [symbol] |
| Proposed Model (28) | ~350k | 90.6 | 75.26 | 78.49 | 79.102 | 97.29 | 80.04 | 59.96 | 80.58 | 71.49 |
| Proposed Model (224) | ~350k | 91.15 | 74.95 | 78.51 | 82.407 | 97.35 | 87.52 | 59.18 | 86.66 | 72.4 |

[symbol]

indicates data missing or illegible when filed

Datasets: Unlike common imaging formats such as RGB images medical image analysis requires the model to interact with data from multiple modalities. To explore the model's ability to generate descriptive representations under multiple data modalities the MedMNISTv2 dataset is used. Sourced from well-known datasets and covering primary data modalities in biomedical imaging, MedMNISTv2 contains a total 708,069 2D medical images divided up into multiple binary and multi-class tasks. Modalities include Pathology, Ultrasound, Chest X-Rays Dermatoscopic, Retinal OCT, Fundus Camera, Blood Cell and Kidney Cortex Microscope data. Due to redundant data modalities within the dataset such as multiple X-Ray and Computed Tomography (CT) of different views of the same patient ChestMNIST, OrganAMNIST and OrganCMNIST are discarded. In addition to medical imaging to showcase the generalizability and scalability of LeavisNet common CV benchmarking datasets such as CIFAR10 and CIFAR100 are used. Finally, to further explore the adaptability of the learned representation for a complex downstream task such as image segmentation, the ISIC 2016 and 2017 skin lesion segmentation dataset may be used.

Model Architecture Variations: The proposed model is modified to the nature of the experimental data use. In particular, for binary and multiclass classification experiments, the raw logits from the model are passed into a single fully connected layer to produce the final output. In the segmentation task the raw logits are passed to a DeepLabv3 classification head to generate the final segmentation mask.

Training Setup: The MedMNISTv2 experiment models are trained using the Adam optimizer with β1=0.9, β2=0.999, a batch size of 128 and an initial learning rate of 1e-3 with a learning rate scheduler. Additionally, simple data augmentation techniques such as color jitter and auto contrast are used with all models in the MedMNISTv2 experiments. With the computer vision benchmarking datasets CIFAR10 and CIFAR100 AutoAugment was used with same Adam optimizer. For the segmentation experiments a modified approach to training was taken which included heavy data augmentations. Example augmentations include Random Brightness Contrast, Elastic Deformations, Grid Distortions and Optical Distortions. To be consistent with the published literature, input image size was constrained to 224×224 for all models tested. No custom preprocessing or custom postprocessing pipelines were used during the segmentation experiments to showcase the reliability of the raw features from the proposed model. All experiments across all neural network architectures included in the experiments use the same seed, Seed 42, to avoid choosing of lucky a seed.

Metrics: For all classification experiments Cross-Entropy Loss was used during training. The segmentation experiments used a Dice Loss as the main loss function.

### MedMINISTv2 Results

Performance Over Multiple Data Modalities: To compare and contrast the performance of the proposed LeavisNet model on the MedMNISTv2 experiments the architecture 300 was benchmarked against some of the most highly cited lightweight and efficient architectures such as EfficientNet, MobileNetv3, MNasNet and SqueezeNet. Furthermore, AutoML solutions such as Auto-Sklearn, AutoKeras and Google AutoM have been included to further demonstrate the performance of LeavisNet. As shown in Table 2, the LeavisNet architecture demonstrates competitive performance across some of the most prominent models in deep learning literature. A wide range of medical imaging modalities are considered within this experiment such as Colon Pathology, Chest X-Ray, Dermatoscopic, Retinal OCT, Ultrasound and Microscopic images. With datasets sizes varying from 100 to 100,000 images it has been demonstrated that the model can generalize without the need for pretraining on much larger datasets. With roughly 350 k parameters, LeavisNet rivals image classification performances of models with parameters 4x to 71x that of LeavisNet.

TABLE 2

| | Parameter | Top 1 | Top 5 |
|---|---|---|---|
| Method/Model | Count | Accuracy | Accuracy |
| Trained and Tested on CIFAR10 | | | |
| MobileNetv3 Large | ~4.2M | 94 | 98.7 |
| MobileNetv3 Small | ~1\|.5M | 90.4 | 98.52 |
| MNasNet | ~3.1M | 91.79 | 98.59 |
| EfficientNet-B0 | ~4M | 91.77 | 98.55 |
| Proposed Model | ~350k | 93.25 | 98.57 |
| Trained and Tested on CIFAR100 | | | |
| MobileNetv3 Large | ~4.2M | 70.74 | 89.95 |
| MobileNetv3 Small | ~1.5M | 66.17 | 87.32 |
| MNasNet | ~3.1M | 67.68 | 90.09 |
| EfficientNet-B0 | ~4M | 71.12 | 91.33 |
| Proposed Model | ~350k | 70.38 | 91.37 |

### CIFAR Results

General Purpose Architecture: To demonstrate LeavisNet's scalability to generalize over a large number of classes, experiments on common CV benchmarking datasets are shown. The CIFAR10 and CIFAR100 datasets are benchmarked against similar highly cited light weight architectures seen in Table 3 to showcase the performance of the proposed architecture. The use of excessively large architectures on small datasets as seen in MedMNISTv2 and CIFAR experiments provide no meaningful understanding towards the need for light weight models in medical deep learning. As seen in Table 3, LeavisNet outperforms the competition in CIFAR10 and is highly competitive in CIFAR100 all with roughly 350 k parameters. This indicates that the model of the present disclosure is capable of learning meaningful representations regardless of the data modality. Thus, it can be used as a drop-in replacement and a general-purpose feature extractor for a multitude of problems involving natural imagery.

TABLE 3

| Method/Model | Dice | Jaccard |
|---|---|---|
| Trained and Tested on ISIC-2016 | | |
| [symbol] [38] | 91 | 84.3 |
| [symbol] [39] | 89 | 82.9 |
| [symbol] [40] | 89.5 | 82.22 |
| [symbol] et al. [41] | 91.18 | 84.64 |
| [symbol] et al. [42] | 91.2 | 84.7 |
| LesionNet [43] | 92.39 | 86.47 |
| [symbol] et al. [30] | 90.39 | 85.96 |
| [symbol] et al. [30] | 88.33 | 84.15 |
| [symbol] et al. [30] | 90.27 | 85.88 |
| Proposed Model | 91.08 | 84.54 |
| Trained and Tested on ISIC-2017 | | |
| [symbol] et al. [42] | 84.9 | 76.5 |
| [symbol] et al. [44] | 84.7 | 76.2 |
| [symbol] et al. [41] | 84.4 | 76 |
| [symbol] et al. [45] | 77 | 62 |
| [symbol] et al. [46] | 87.08 | 77.11 |
| [symbol] et al. [47] | 85.8 | 77.2 |
| LesionNet [43] | 87.87 | 78.2 |
| [symbol] et al. [30] | 90.39 | 80.05 |
| [symbol] et al. [30] | 79.28 | 77.66 |
| [symbol] et al. [30] | 82.96 | 80.03 |
| Proposed Model | 83.51 | 74.54 |

[symbol]

indicates data missing or illegible when filed

### Segmentation Results

An indicator for a robust deep learning architecture is its ability to learn meaningful representations of any given input. For LeavisNet, it is capable of extracting features across variety of medical imaging formats including natural images. To demonstrate the adaptability of the representations learned by LeavisNet to downstream deep learning tasks, consider the ISIC 2016 and ISIC 2017 datasets. The ISIC segmentation experiments are compared against the current state of the art architectures which includes LesionNet and UNet variants. Due to the unique nature of the skin lesion segmentation which includes sources of noise such as hair, many of the solutions proposed for skin lesion segmentation requires multiple pre and post processing stages prior to generating the final outputs. In addition to pre and post processing stages, each of the models which includes the current state of the art solutions are very large models with an excessive of 50+M parameters. As shown in Table 3, the architecture of the present disclosure generates competitive results to each of the models by combining LeavisNet with the DeepLab Segmentation Head generating an efficient segmentation model with a parameter count of 2.2 M.

The construction and arrangement of the systems and methods as shown in the various implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes¬from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

1. An autonomous ultrasonographic imaging device comprising:
an ultrasound transducer array;
a processor; and
memory having instructions stored thereon that, when executed by the processor, cause the ultrasonographic imaging device to:
capture, using the ultrasound transducer array, a plurality of ultrasound images of a target area of a subject;
evaluate the plurality of ultrasound images using an artificial intelligence (AI) model to detect a medical condition and guide an operator to position the ultrasound imaging device; and
display, via a user interface, an indication of whether the medical condition was detected.

2. The autonomous ultrasonographic imaging device of claim 1, further comprising an electromechanical movement assembly, wherein the ultrasound transducer array is coupled to the electromechanical movement assembly such that the positioning of the ultrasound transducer array is adjustable.

3. (canceled)

4. The autonomous ultrasonographic imaging device of claim 2, wherein to capture the plurality of images, the instructions further cause the autonomous ultrasonographic imaging device to control the electromechanical movement assembly to incrementally adjust the positioning of the ultrasound transducer array between captures of each of the plurality of ultrasound images.

5. The autonomous ultrasonographic imaging device of claim 4, wherein the electromechanical movement assembly is configured to incrementally rotate the ultrasound transducer array about an axis between captures of each of the plurality of ultrasound images to generate a rotating cross-section of the target area of the subject.

6. (canceled)

7. (canceled)

8. The autonomous ultrasonographic imaging device of claim 1, wherein to display the indication of whether the medical condition was detected:
a control indicator light in the user interface is turned on and a display is turned on if the medical condition is detected;
the control indicator light in the user interface is turned on and the display is turned off if the medical condition is not detected; or
the control indicator light in the user interface is turned off and the display is turned on if results of the evaluation of the plurality of images are determined to be invalid.

9. The autonomous ultrasonographic imaging device of claim 1, wherein the user interface further comprises a speaker for providing audible feedback to an operator.

10. The autonomous ultrasonographic imaging device of claim 9, wherein the instructions further cause the autonomous ultrasonographic imaging device to output a sound via the speaker when the medical condition is detected or once the evaluation of the plurality of ultrasound images is complete.

11. The autonomous ultrasonographic imaging device of any of claim 1, wherein the AI model is one of a machine learning model, a deep learning model and a convolutional neural network.

12. (canceled)

13. (canceled)

14. The autonomous ultrasonographic imaging device of claim 11, wherein the deep learning model comprises a feature extraction module, a convolutional layer module, and a transformer module.

15. The autonomous ultrasonographic imaging device of any of claim 1, wherein the autonomous ultrasonographic imaging device is configured for ocular ultrasound imaging and is worn or positioned over an eye of the subject.

16. (canceled)

17. The autonomous ultrasonographic imaging device of claim 15, wherein the plurality of ultrasound images are evaluated, in part, to determine an optical nerve sheath diameter (ONSD).

18. The autonomous ultrasonographic imaging device of claim 1, wherein the autonomous ultrasonographic imaging device is configured to be worn on a chest of the subject.

19. The autonomous ultrasonographic imaging device of claim 1, wherein the ultrasound transducer array comprises at least one ultrasound transducer, the at least one ultrasound transducer comprising a lens, a matching layer, a ground electrode, a piezoelectric ceramic layer, a signal electrode, and an acoustic absorption layer.

20. The autonomous ultrasonographic imaging device of claim 1, the instruction further causing the autonomous ultrasonographic imaging device to receive programming instructions from a remote device using via an application programming interface (API), wherein the programming instructions define the medical condition that the plurality of images are evaluated to detect and/or include the AI model.

21. (canceled)

22. A method of detecting medical conditions using an autonomous ultrasonographic imaging device, the method comprising:
capturing, by an ultrasound transducer array of the autonomous ultrasonographic imaging device, a plurality of ultrasound images of a target area of a subject, wherein a position of the ultrasound transducer array is automatically and incrementally adjusted between captures of each of the plurality of images;
evaluating the plurality of ultrasound images using an artificial intelligence (AI) model to detect a medical condition and guide an operator to position the ultrasound imaging device; and
displaying, via a user interface of the autonomous ultrasonographic imaging device, an indication of whether the medical condition was detected.

23. The method of claim 22, wherein the position of the ultrasound transducer array is automatically and incrementally adjusted by an electromechanical movement assembly, wherein the ultrasound transducer array is coupled to the electromechanical movement assembly.

24. The method of claim 23, wherein capturing the plurality of images further comprises controlling the electromechanical movement assembly to automatically and incrementally adjust the positioning of the ultrasound transducer array.

25. The method of claim 24, wherein the electromechanical movement assembly is configured to incrementally rotate the ultrasound transducer array about an axis between captures of each of the plurality of ultrasound images to generate a rotating cross-section of the target area of the subject.

26. (canceled)

27. (canceled)

28. The method of claim 22, wherein displaying the indication of whether the medical condition was detected comprises:
turning on a control indicator light in the user interface and a display if the medical condition is detected;
turning on the control indicator light in the user interface and turning off the display if the medical condition is not detected; or
turning off the control indicator light in the user interface and turning on the display if results of the evaluation of the plurality of images are determined to be invalid.

29. The method of claim 22, wherein the user interface further comprises a speaker for providing audible feedback to an operator.

30. The method of claim 29, further comprising outputting a sound via the speaker when the medical condition is detected or once the evaluation of the plurality of ultrasound images is complete.

31. (canceled)

32. (canceled)

33. (canceled)

34. (canceled)

35. The method of claim 22, wherein the autonomous ultrasonographic imaging device is configured for ocular ultrasound imaging and is worn or positioned over an eye of the subject.

36. (canceled)

37. The method of claim 35, wherein the plurality of ultrasound images are evaluated, in part, to determine an optical nerve sheath diameter (ONSD).

38. (canceled)

39. The method of claim 22, wherein the ultrasound transducer array comprises at least one ultrasound transducer, the at least one ultrasound transducer comprising a lens, a matching layer, a ground electrode, a piezoelectric ceramic layer, a signal electrode, and an acoustic absorption layer.

40. The method of claim 22, further comprising receiving programming instructions from a remote device using via an application programming interface (API), wherein the programming instructions define the medical condition that the plurality of images are evaluated to detect and/or include the AI model.

41. (canceled)
